(19) United States
(12) Patent Application Publication
(10) Pub. No.: US 2020/0330229 A1
(43) Pub. Date: Oct. 22, 2020

(54) MITRAL BRACE

(71) Applicant: TEL HASHOMER MEDICAL RESEARCH, INFRASTRUCTURE AND SERVICES LTD., Ramat Gan (IL)

(72) Inventors: Alain Elie SERRAF, Jerusalem (IL); Mila Shai Raziel TEJMAN-YARDEN, Tel Aviv (IL); Roey SHAFRIR, Modi'in, Makabim-Re'ut (IL)

(21) Appl. No.: 16/764,012

(22) PCT Filed: Nov. 14, 2018
(86) PCT No.: PCT/IL2018/051227

§ 371 Date: May 14, 2020

Related U.S. Application Data
(60) Provisional application No. 62/586,197, filed on Nov. 15, 2017.

(51) Int. Cl. A61F 2/24; A61B 17/00; A61B 17/064; A61B 17/072

(52) U.S. Cl. CPC A61F 2/2463; A61F 2/2466; A61B 17/00234; A61B 17/0644; A61F 2220/0066; A61B 2017/00243; A61B 2017/00867; A61F 2220/0083; A61B 17/07292

(57) ABSTRACT

Apparatus for supporting a leaflet of a cardiac valve having an annulus and a plurality of leaflets extending from the annulus, the apparatus comprising: at least one support brace mountable to the cardiac valve and having: first and second ends each configured to be mounted to the cardiac valve in a region near a commissure of the cardiac valve associated with a leaflet of the plurality of leaflets; and a body configured to be attached to the leaflet and extend between the first and second ends along a lip of the leaflet.

### RELATED APPLICATIONS

This application claims benefit under 35 U.S.C. 119(e) of U.S. Provisional Application 62/586,197, filed Nov. 15, 2017, the disclosure of which is incorporated herein by reference.

### FIELD

Embodiments of the disclosure relate to reinforcing dynamic shape of a cardiac valve to improve functioning of the valve in controlling flow of blood in the heart.

### BACKGROUND

The human heart comprises two blood pumps that operate in synchrony to oxygenate and deliver oxygenated blood to the body. A first pump receives deoxygenated blood from the various parts the body and pumps the blood through the lungs to be oxygenated. The second pump receives the oxygenated blood from the lungs and pumps it to flow through the blood vessels of the circulatory system and deliver oxygen and nutrients to the body parts. The two pumps are located adjacent each other in the heart and each pump comprises two chambers, an atrium that receives blood and a ventricle that pumps blood.

The first pump is located on the right side of the heart and comprises the right atrium and right ventricle. The second pump is located on the left side of the heart and comprises the left atrium and left ventricle of the heart.

In the first pump, deoxygenated blood enters the right atrium, and during a diastolic part of the heart cycle the right ventricle is relaxed and the blood flows from the right atrium through a tricuspid valve into the right ventricle. The right ventricle contracts during the systolic part of the heart cycle to pump the deoxygenated blood that it receives from the right atrium out of the ventricle through a pulmonary valve and into the pulmonary artery for oxygenation in the lungs. The tricuspid and pulmonary valves control direction of blood flow in the right side of the heart. The tricuspid valve, for example, opens to let deoxygenated blood flow from the right atrium into the right ventricle and closes to prevent deoxygenated blood from regurgitating into the right atrium when the right ventricle contracts.

In the second pump the left atrium receives oxygenated blood from the lungs via pulmonary veins. Oxygenated blood flows from the left atrium into the left ventricle during diastole via a bicuspid valve referred to as the mitral valve. During systole the left ventricle contracts to pump the oxygenated blood that it receives from the left atrium out of the heart through an aortic valve and into the aorta for delivery to the body. The mitral and aortic valves operate to control direction of blood flow in the left side of the heart. For example, the mitral valve opens during diastole to enable blood to flow from the left atrium to the left ventricle and closes to prevent regurgitation of oxygenated blood from the left ventricle to the left atrium during systole when the left ventricle contracts to pump oxygenated blood into the aorta.

Each cardiac valve comprises a set of matching “flaps”, also referred to as “leaflets” or “cusps”, which are mounted to and extend from a supporting ring structure of fibrous tissue, referred to as the annulus of the valve. The leaflets are configured to align and overlap each other, or coapt, along free edges of the leaflets to close the valve and prevent undesired, retrograde blood flow when a blood pressure gradient across the valve increases during cardiac systole. The valve opens and the free edges part when the leaflets are pushed apart from each other by a gradient in blood pressure across the valve that operates to generate antegrade blood flow in a desired, antegrade direction through the valve during cardiac diastole.

Efficient cardiac function is complex and dependent upon orchestrated cooperation of many different features of the heart and proper functioning of the heart's valves. Proper functioning of a cardiac valve may become compromised by disease or injury to the valve's leaflets, the valve annulus, and/or chordae that prevent prolapse of the leaflets. The damage may result in failure of the leaflets to coapt properly, causing regurgitation and poor cardiac function. Damage may be so severe as to warrant surgical intervention to effect repair or replacement of the valve and provide a person suffering from cardiac malfunction with an acceptable state of health and quality of life.

### SUMMARY

An aspect of an embodiment of the disclosure relates to providing apparatus, hereinafter also referred to as a cardiac valve support (CVS) that may be coupled to at least one leaflet of a cardiac valve to configure and/or support shape of the at least one leaflet and improve dynamic functioning of the at least one leaflet and the valve. Optionally, the CVS is configured to be coupled to a leaflet or leaflets of the mitral valve of the heart.

In an embodiment a CVS may comprise at least one support brace that couples to a leaflet of a cardiac valve of a heart to configure and/or support shape of the leaflet and aid in preventing or mitigating prolapse of the leaflet into the heart's atrium during systole or through a semilunar valve from a great artery to its related ventricle during diastole. In an embodiment, the at least one support brace, hereinafter also referred to as a leaflet brace, is configured to be coupled to the annulus of the valve and to extend between regions of the annulus near to commissures of the valve along a free edge, also referred to as “lip”, of the leaflet. Optionally, the support brace is wire or band shaped. In an embodiment, a support brace comprises a plurality of independent elements, hereinafter also referred to as leaflet staples, that may be coupled to a cardiac valve leaflet and be daisy chained one to the other along a region of the lip of the leaflet. In an embodiment, a support brace, also referred to as a “spoke brace”, comprises a backbone anchor configured to be coupled to the annulus of the valve and a plurality of spokes that extend from the backbone anchor configured to support an area of the leaflet.

In an embodiment of the disclosure a CVS comprises at least first and second support braces that couple respectively to the posterior and anterior leaflets of a mitral valve. The braces cooperate to limit motion of the leaflets and reduce or prevent regurgitation of blood from the left ventricle into the left atrium during systole. The first support brace, hereinafter also referred to as a posterior leaflet brace, is optionally coupled to the mitral valve annulus and extends between regions of the commissures of the mitral valve along the lip of the posterior leaflet. The second support brace, hereinafter also referred to as an anterior leaflet brace, is optionally coupled to the mitral valve annulus to extend between regions of the commissures of the mitral valve along the lip of the anterior leaflet. The anterior leaflet brace or a portion thereof may be located on an antegrade, or ventricle side of the posterior leaflet brace so that the posterior leaflet brace operates as a motion limiter, or “stop”, for the anterior leaflet to prevent or moderate prolapse of the anterior leaflet into the left atrium during systole and improve functioning of the mitral valve. In an embodiment, the posterior leaflet brace may comprise at least one stop spur that protrudes from the posterior leaflet brace to engage the anterior leaflet brace during systole and aid in limiting prolapse of the anterior leaflet.

This summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1 schematically shows a cross section of a human heart that displays the heart chambers and cardiac valves of the heart;

FIG. 2A schematically shows a cutaway perspective view of a human heart that provides a perception of the three-dimensional structure of the mitral and tricuspid valves of the heart;

FIG. 2B shows a schematic plan view of the mitral valve as seen from the atrium of the heart shown in FIGS. 1 and 2A;

FIG. 3A schematically shows a CVS comprising a single anterior leaflet brace configured to be attached to the annulus of a mitral valve and formed having periodic undulations, in accordance with an embodiment of the disclosure;

FIG. 3B schematically shows a CVS configured to be attached to the anterior leaflet of a mitral valve absent attachment to the annulus of the valve, in accordance with an embodiment of the disclosure;

FIGS. 3C-3E schematically show CVSs comprising an anterior leaflet brace formed having different respective patterns of undulations, in accordance with embodiments of the disclosure;

FIG. 3F is a photograph of a torn anterior leaflet of a mitral valve in a pig heart;

FIG. 3G is a photograph of a CVS similar to that shown in FIG. 3A surgically attached to the mitral valve's torn anterior leaflet shown in FIG. 3E to reconstruct the leaflet and improve valve function, in accordance with an embodiment of the disclosure;

FIG. 3H is a photograph of the mitral valve and CVS shown in FIG. 3F exhibiting improved leaflet coaptation and valve function resulting from deployment of the CVS, in accordance with an embodiment of the disclosure;

FIG. 4A schematically shows a CVS comprising an anterior leaflet brace and a posterior leaflet brace in accordance with an embodiment of the disclosure;

FIG. 4B and FIG. 4C schematically show CVSs comprising two anterior leaflet braces and a single posterior leaflet brace in accordance with embodiments of the disclosure;

FIG. 5 schematically shows a hinged CVS comprising an anterior leaflet brace flexibly hinged to a posterior leaflet brace in accordance with an embodiment of the disclosure;

FIG. 6 schematically shows a CVS comprising an anterior leaflet brace and a posterior leaflet brace having stop spurs operable to limit motion of the anterior leaflet brace, in accordance with an embodiment of the disclosure;

FIG. 7 schematically shows a CVS comprising an anterior leaflet link sausage brace comprising a plurality of relatively thick lengths of a bracing material connected by relatively short, thin links in accordance with an embodiment of the disclosure;

FIG. 8A schematically shows a CVS comprising a plurality of leaflet staples supporting an anterior leaflet of a mitral valve in accordance with an embodiment of the disclosure;

FIGS. 8B-8E schematically show top and side views of leaflet staples in accordance with embodiments of the disclosure;

FIG. 8F schematically shows a leaflet support comprising a plurality of wireform staples in accordance with an embodiment of the disclosure;

FIG. 9A schematically shows a CVS comprising a spoke brace having a backbone anchor and spokes supporting an anterior leaflet of a mitral valve in accordance with an embodiment of the disclosure;

FIG. 9B schematically shows the spoke brace shown in FIG. 9A in a delivery configuration secured in a delivery catheter that may be used to deploy the spoke brace, in accordance with an embodiment of the disclosure; and

FIG. 10, schematically shows the spoke brace shown in FIG. 9A in which the spokes function as a scaffolding for a leaflet web, that operates to improve functioning of the anterior leaflet of the mitral valve in accordance with an embodiment of the disclosure.

### DETAILED DESCRIPTION

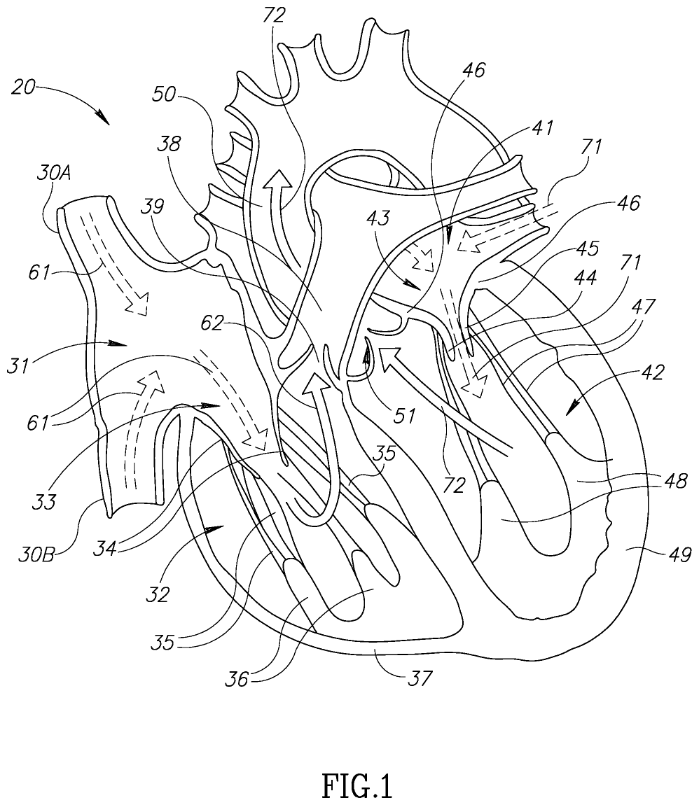

FIG. 1 shows a schematic, stylized cross section of a human heart 20 having a right atrium 31 and a right ventricle 32 that communicate via a tricuspid valve 33 and a left atrium 41 and left ventricle 42 that communicate via a mitral valve 43. Tricuspid valve 33 has three “semilunar” leaflets 34, only two of which are shown in FIG. 1, that are tied by chordae tendineae 35 and papillary muscles 36 to a wall 37 of the right ventricle. Right ventricle 32 communicates with the pulmonary artery 38 via a pulmonary valve 39. Mitral valve 43 has two leaflets, an anterior leaflet 44 in continuity with the wall of the aorta and a posterior leaflet 45. The anterior and posterior leaflets are supported and extend from a mitral annulus 46. Mitral valve leaflets 44 and 45 are respectively tied by chordae tendineae 47 and papillary muscles 48 to a ventricle wall 49. The left ventricle communicates with the aorta 50 via the aortic valve 51.

Deoxygenated blood returning from parts of the body enters right atrium 31 via the superior vena cava 30A and inferior vena cava 30B. The deoxygenated blood passes through tricuspid valve 33 to enter right ventricle 32 during diastole when the right ventricle is relaxed and pressure differential across the tricuspid valve separate leaflets 34 of the tricuspid valve (as schematically shown in FIG. 1) to open the tricuspid valve to blood flow. Flow of deoxygenated blood into the right atrium via the superior and inferior venae cavae 30A and 30B and through tricuspid valve 33 into the right ventricle is schematically indicated by dashed line block arrows 61. During systole right ventricle 32 contracts to pump the deoxygenated blood through pulmonary valve 39 and into pulmonary artery 38 for delivery to the lungs. During systole pressure differential across the tricuspid valve pushes leaflets 34 of tricuspid valve 33 to coapt and close tricuspid valve 33 to blood flow to prevent deoxygenated blood pumped by the right ventricle from regurgitating into the right atrium. Flow of deoxygenated blood pumped by right ventricle 32 into pulmonary artery 38 is schematically indicated by a solid line block arrow 62.

Oxygenated blood from the lungs enters left atrium 41 and passes through mitral valve 43 to enter left ventricle 42 during diastole when the left ventricle relaxes and pressure differential across the mitral valve causes leaflets 44 and 45 to separate to open the mitral valve to blood flow. Flow of oxygenated blood into the left atrium and through mitral valve 43 into the left ventricle is schematically indicated by dashed block arrows 71. During systole left ventricle 42 contracts to pump the oxygenated blood through the aortic valve 51 and into the aorta 50 for delivery to the body. During systole pressure differential across the mitral valve pushes leaflets 44 and 45 to coapt and close mitral valve 43 to blood flow to prevent oxygenated blood pumped by the left ventricle from regurgitating into the left atrium.

Valves 33, 39, 43, and 51 operate to direct flow of blood in the heart and out from the heart, and proper and efficient function of the valves are required to maintain a person's health and quality of life. Various different disease processes may result in damage to a heart valve and compromise valve functioning. For example, functioning of the mitral valve may be compromised by various degrees of stenosis, calcification, distortion of the mitral valve annulus, torn chordae tendineae, and faulty left ventricle functioning. Valve dysfunction and concomitant regurgitation may become so severe as to warrant surgical intervention to provide a person with an acceptable state of health and quality of life.

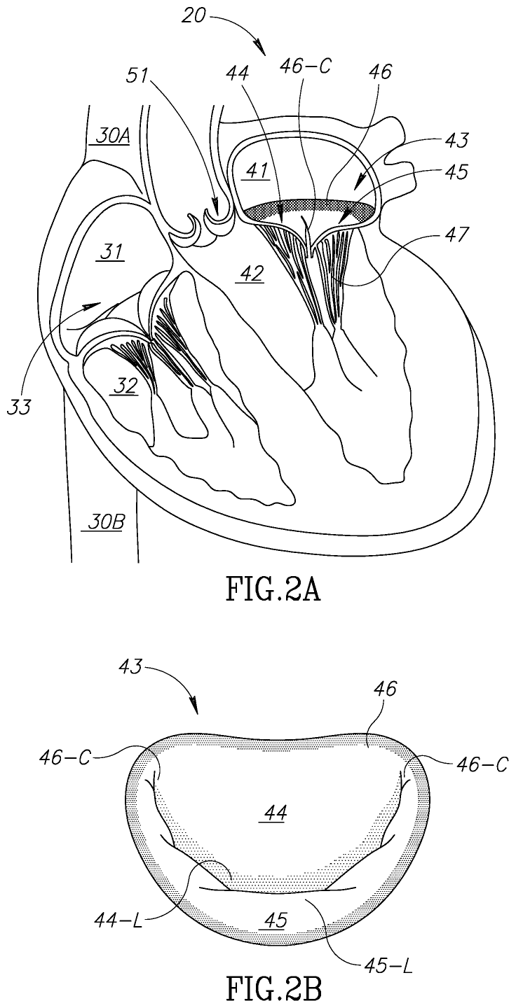

FIG. 2A schematically shows a cutaway perspective view of a human heart 20 that provides a perception of the three-dimensional structure of mitral valve 43 and tricuspid valve 33. In the figure, a portion of annulus 46 of mitral valve 43 that supports mitral valve anterior and posterior leaflets 44 and 45 is shown shaded, and a region of a commissure of the two commissures 46-C at which the leaflets come together is indicated. FIG. 2B schematically shows a schematic plan view of mitral valve 43 as seen from left atrium 42 of heart 20 that shows anterior and posterior leaflets 44 and 45 respectively, annulus 46, and commissures 46-C of mitral valve 43. FIG. 2B also shows lips 44-L and 45-L of anterior and posterior leaflets 44 and 45 respectively.

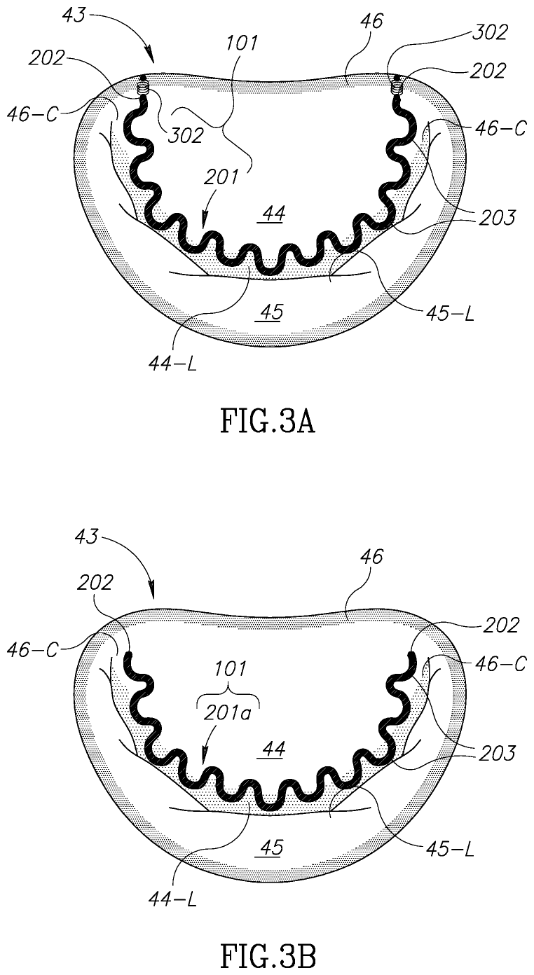

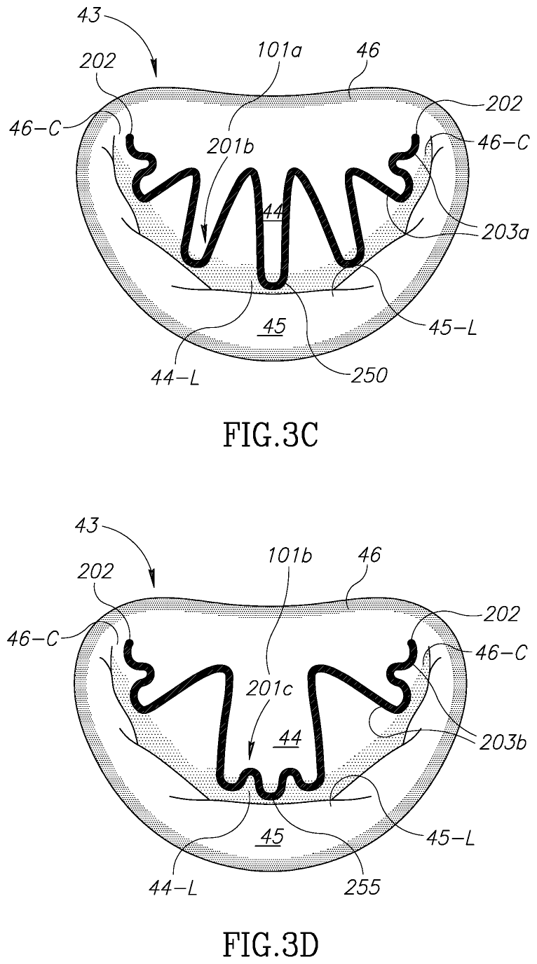

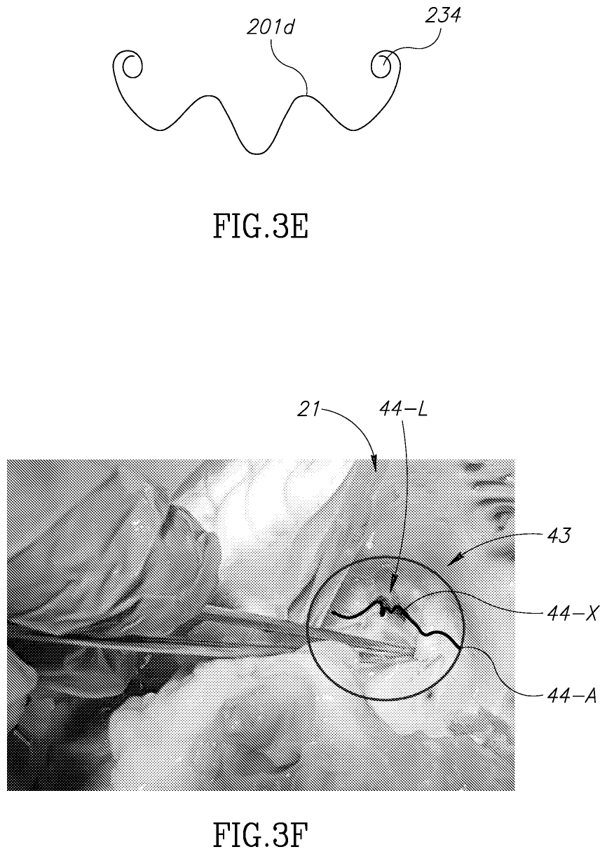

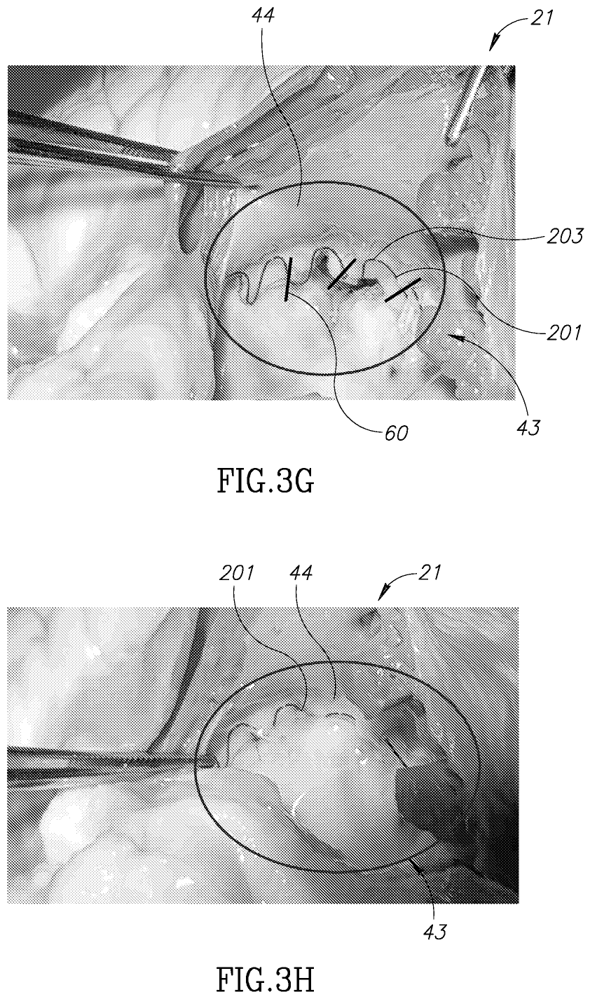

FIG. 3A schematically shows a CVS 101 operable to provide support for a mitral valve 43 in accordance with an embodiment of the disclosure. CVS 101 optionally comprises an anterior leaflet brace 201 having ends 202 that may be mounted to annulus 46, optionally by coil springs 302, in respective regions of the annulus near commissures 46-C and be shaped to lie along lip 44-L of anterior leaflet 44 of mitral valve 43. Optionally, CVS 101 is sized so that distances of ends 202 to respective commissures 46-C is between about 2 mm to about 3 mm. Leaflet brace 201 may be formed as a wire or band shaped to lie along lip 44-L of anterior leaflet 44 of the mitral valve and, optionally, having undulations 203. The undulations may be substantially periodic and have a spatial amplitude and spatial wavelength configured to ameliorate deformities in the mitral valve anterior leaflet.

By way of example, undulations 203 may have spatial amplitude and/or wavelength greater than 3 mm (millimeters). Optionally the amplitude and/or wavelength is greater than 5 mm. The width of the support brace may be about 0.25 mm. In an embodiment, leaflet brace 201 may be formed from any suitable biocompatible material, which may by way of example comprise a metal, a polymer, or composite material, and optionally be a shape memory material such as Nitinol.

Leaflet brace 201 in an embodiment, may have different properties at different locations along a length of leaflet brace 201 between ends 202. Properties of support brace 201 that may change depending on location are, by way of example, any one or any combination of more than one of the following: contour, material composition of the support brace material, cross-section size, and/or shape. Undulations 203 and/or properties of a leaflet brace 201 may be configured to match to support brace 201 to compensate for a particular deformity and/or abnormality of mitral valve 43

Leaflet brace 201 may be attached, to the atrial side, of annulus 46 and/or anterior leaflet 44 by sutures, staples, and/or by self-deforming shape memory material tangs (not shown) that are configured to transform from a martensite state to an austenite state during which the tangs curl to puncture and grip tissue of annulus 46 and/or anterior leaflet 44. In an embodiment leaflet brace 201 may be attached to annulus 46 and/or anterior leaflet 44 by “ring” loops (not shown) that may be mounted to the annulus and/or leaflet through which leaflet brace 201 is threaded. Leaflet brace 201 may be deployed by an open-heart or a transcatheter surgical procedure. In an embodiment, leaflet brace 201 may be attached along lip 44-L of leaflet 44 without being attached to annulus 46 of mitral valve 43.

FIG. 3B schematically shows a CVS 101 comprising an anterior leaflet brace 201a, optionally having undulations 203. Leaflet brace 201a is shaped to lie along lip 44-L and have ends 202 that may be attached to respective regions of anterior leaflet 44 near commissures 46-C rather than to annulus 46.

A CVS in accordance with an embodiment may exhibit undulations that vary in spatial amplitude and/or spatial wavelength. The spatial amplitude and spatial wavelength of undulations may optionally be configured to match and ameliorate a deformity and or abnormality in a valve leaflet. mitral valve anterior leaflet 44.

By way of example, FIG. 3C schematically shows a CVS 101a mounted to mitral valve anterior leaflet 44 and comprising a leaflet brace 201b having undulations 203a that vary in spatial amplitude and spatial wavelength. Optionally, undulations 203a of leaflet brace 201b, tend to increase in spatial amplitude with distance from each end 202 toward a location of a same maximum amplitude in the undulations. In an embodiment as shown in FIG. 3C for leaflet brace 201b, the maximum amplitude is located substantially at a central region of the support brace or substantially at a location where anterior leaflet 44 is widest.

FIG. 3D schematically illustrates a leaflet brace 201c having undulations 203b that vary in spatial amplitude and wavelength. In an embodiment, undulations 203 to 203b of leaflet braces 201 to 201c are not limited to the number of undulations presented in FIGS. 3A to 3G but may have any plurality of undulations. FIG. 3E schematically illustrates a leaflet brace 201d having a variation of undulations and eyelets 234. Eyelets 234 may be advantageous for attaching the leaflet brace to the leaflet. In an embodiment, eyelets 234 may be formed of shape memory material which may be used for shape deforming deployment of leaflet brace 201d.

FIGS. 3F-3H, are pictures acquired of different stages in an experiment undertaken to investigate deployment and functioning of a leaflet brace 201 in accordance with an embodiment of the disclosure. The leaflet brace was deployed to a torn leaflet 44 of a mitral valve 43 in an excised pig heart 21 shown prior to deployment of the leaflet brace from an atrial view in FIG. 3F. In FIG. 3F the tear in leaflet 44 is indicated by a dark region 44-X along lip 44-L of the leaflet indicated schematically by a line 44-A drawn along the lip.

FIG. 3G depicts leaflet brace 201 having undulations 203 surgically attached to the mitral valve's torn anterior leaflet 44, shown in FIG. 3F. The attachment of leaflet brace 201 as shown in FIG. 3F, of anterior leaflet 44 is along lip 44-L of the leaflet. Sutures, schematically represented by short line segments 60, attach leaflet brace 201 to leaflet 44.

After attachment of leaflet brace 201, saline was injected into the left ventricle of heart 21, and the left ventricle was manually compressed to investigate the mitral valve functioning after deployment of brace 201 to support leaflet 44 during the systolic phase of the cardiac cycle. FIG. 3H is an image depicting the reconstructed leaflet having leaflet coaptation, and valve function resulting from deployment of leaflet brace 201. As heart 21 is manually compressed both leaflets of mitral valve 43 bulge slightly into the atrium, and satisfactorily coapt and prevent fluid, saline solution noted above, regurgitation into the atrium. Mitral valve 43 regained competence.

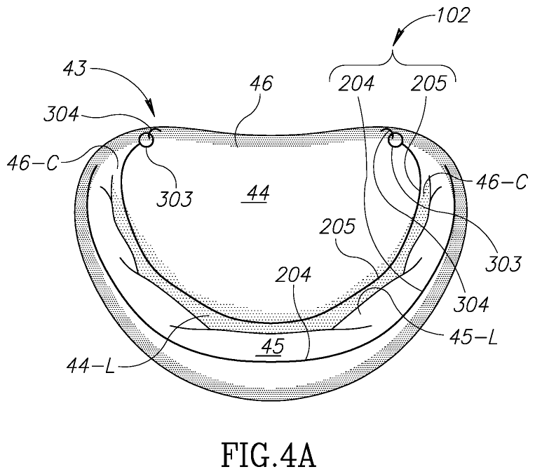

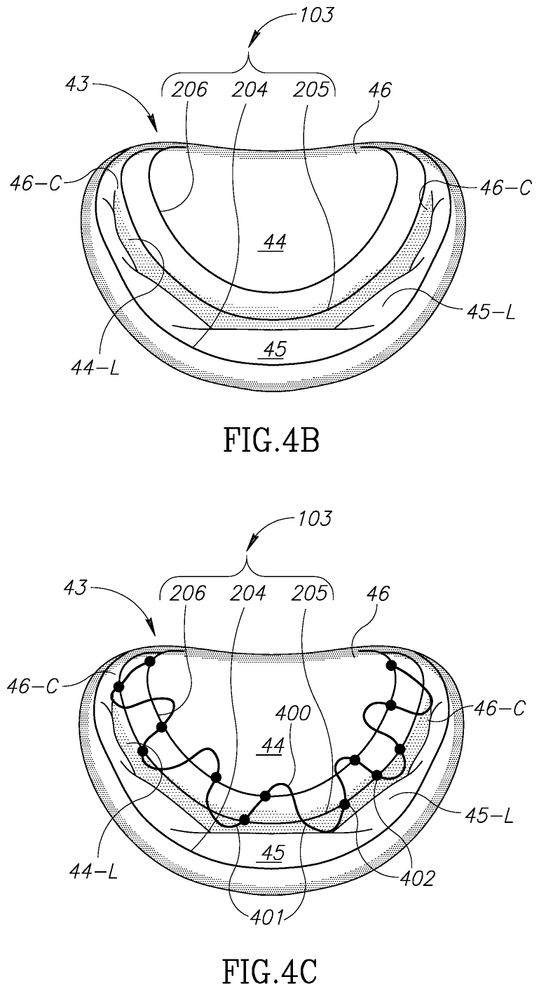

FIG. 4A schematically shows a CVS 102 comprising a posterior leaflet brace 204 and an anterior leaflet brace 205 configured to support and maintain shape respectively of a posterior leaflet 45 and an anterior leaflet 44 of a mitral valve 43, in accordance with an embodiment of the disclosure. Optionally, anterior leaflet brace 205 is mounted to annulus 46 by eyelets 303 that are attached to or formed at ends of leaflet brace 205 and loop through “ring” loops 304 that pierce and anchor to the annulus near to commissures 46-C. Leaflet brace 205 may extend from ring loops 304 along the atrial side of lip 44-L of anterior leaflet 44. In an embodiment, posterior leaflet brace 204 is also mounted to annulus 46, optionally by eyelets and loops similarly to the way in which leaflet brace 205 is mounted to the annulus and extends along posterior leaflet 45 between regions of the annulus near commissures 46-C. In an embodiment, braces 204 and/or 205 may be attached to leaflets 44 and/or 45 along their respective lips 44-L and 45-L optionally by any of the methods and devices by which leaflet brace 201 may be attached to a leaflet. In an embodiment, braces 204 and/or 205 may be attached to leaflets 44 and/or 45 without being attached to annulus 46 of mitral valve 43.

For example, leaflet braces 204 and 205 may be attached to, optionally, the atrial side of leaflets 45 and 44 by sutures, self-deforming shape memory tangs (not shown) and/or loops. Leaflet braces 204 and 205 may be formed from any suitable biocompatible, optionally shape memory, material. Leaflet braces 204 and 205 may be deployed by an open-heart surgical procedure.

FIG. 4B schematically shows a CVS 103 similar to CVS 102 but comprising in addition to leaflet braces 204 and 205, at least one more anterior leaflet brace 206 mounted between annulus 46 and leaflet brace 205 and configured to extend along anterior leaflet 44. Optionally, leaflet brace 206 is attached to annulus 46 and/or leaflet 44 in any of the manners and/or with any of the devices by which leaflets 204 and 205 may be attached to a structure of mitral valve 43.

FIG. 4C schematically shows a CVS 103.1 that is a modification of CVS 103 that optionally includes all the elements of CVS 103, but in addition comprises a tying brace 400 that connects leaflet brace 205 to leaflet brace 206. Optionally, tying brace 400 comprises a series of undulations 401 that span a distance between leaflet braces 205 and 206 and is connected to the leaflet braces at a plurality of optional junctions 402. In an embodiment, at least one undulation 401, of the series of tying brace 400, extends beyond lip 44-L of anterior leaflet 44. Optionally, a portion of the at least one undulation 401, at its peak, extends beyond lip 44-L and lies on the antegrade side of posterior leaflet brace 204 so that it butts up against leaflet brace 204 during systole.

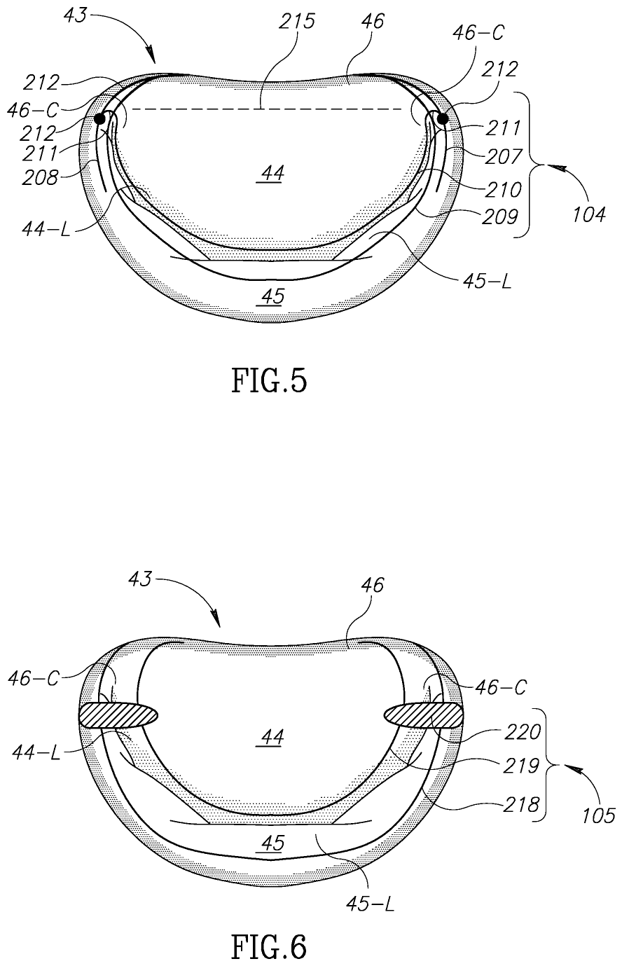

FIG. 5 schematically shows a CVS 104 comprising first and second side bands 207 and 208 that are coupled to posterior and anterior leaflet braces 209 and 210 in accordance with an embodiment of the disclosure. Side bands 207 and 208 are configured to be mounted to regions of annulus 46 near to commissures 46-C of a mitral valve 43 and to follow contour of annulus 46 in the vicinities of the commissures 46-C to which they are respectively mounted, in accordance with an embodiment. The side bands may be mounted to annulus 46 by sutures, staples, self-deforming shape memory tangs (not shown) and/or ring loops. Posterior and anterior leaflet braces 209 and 210 may be fastened to posterior and anterior leaflets 45 and 46 by any suitable means such as by way of example, sutures, shape memory tangs, and/or ring loops.

Anterior leaflet brace 210 is connected to side bands 207 and 208 by coupling loops 211 that join the side bands at joints 212 that allow anterior leaflet brace 210 to rotate relatively freely about an axis schematically indicated by a dashed line 215 through an angle of rotation consistent with a desired range of motion of anterior leaflet 44 during a cardiac cycle. A joint 212 may be any of various types of joints that allow the desired range of motion for anterior leaflet 44. For example, a joint 212 may be a hinge joint, a swivel joint comprising a shaft and join or eyelet and loop, a socket joint, or an elastic joint which may comprise a coil spring or elastic bending beam or rod. Coupling loops 211 are located below the posterior leaflet brace 209. Since amplitude of motion of posterior leaflet 45 during a cardiac cycle is relatively small and width of posterior leaflet 45 is relatively small, amplitude of motion of posterior leaflet brace 209 is relatively limited and contact of coupling loops 211 with posterior leaflet brace 209 operates to limit motion of anterior leaflet brace 210 in a retrograde direction and thereby prolapse of the anterior leaflet.

FIG. 6 schematically shows a CVS 105 comprising posterior and anterior leaflet braces 218 and 219 and comprising stop spurs 220 mounted to the posterior leaflet brace near commissure 46-C of mitral valve 43 in accordance with an embodiment of the disclosure. Stop spurs 220 are configured to extend over a portion of the anterior leaflet brace 219. The posterior and anterior leaflet braces are mounted to annulus 46 and respectively to the posterior and anterior leaflets by sutures, staples, shape memory tangs, and/or ring loops. Posterior leaflet brace 218 may be mounted along its length close to or to annulus 46. Stop spurs 20 operate to limit motion of anterior leaflet brace 219 and thereby prolapse of anterior leaflet 44 to which the anterior leaflet brace is attached.

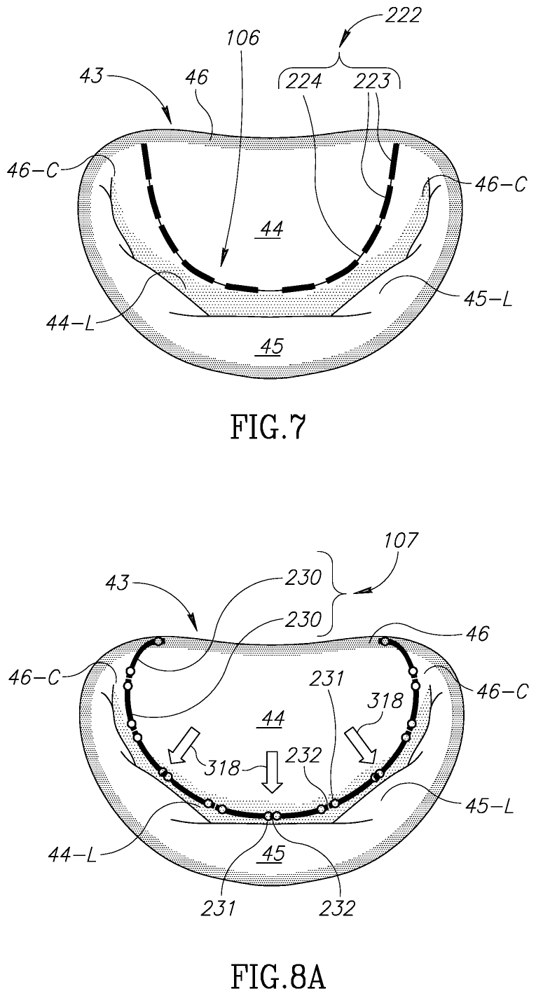

FIG. 7 schematically shows a CVS 106 comprising a leaflet link brace 222, reminiscent of a length of sausage links, comprising relatively thick segments 223, also referred to as “support links”, of a biocompatible bracing material connected by relatively short thin connecting segments 224. Optionally, as shown in FIG. 7, link brace 222 may be attached to annulus 46 and anterior leaflet 44 to support the leaflet, in accordance with an embodiment of the disclosure. Link brace 222 is readily bent to adapt the shape of the link brace to deformities of a cardiac valve leaflet 44 to alleviate different leaflet and cardiac valve deformities. Optionally, each of a plurality of support links 223 is formed having tangs (not shown) configured to puncture leaflet 44 and bend to anchor the leaflet link brace 222 to leaflet 44. Optionally, the tangs are formed from a shape memory material.

Links 223 may be between about 4 mm to about 8 mm long and having a maximum cross-section dimension between about 3 mm to 5 mm. In an embodiment, link brace 222 may comprise a polymer sleeve (not shown) that covers each link 223 and optionally connects the links. The polymer sleeve and connecting segments 224 may be cut between links 223 to modify the length of link brace 222. In an embodiment a CVS similar to CVS 106 may comprise a link brace which is absent connecting segments 224 and in which a polymer sleeve serves to connect links 223. It is noted that whereas a polymer sleeve is used specifically to links 223 on a leaflet, any leaflet brace in accordance with any embodiment of this disclosure may have a polymer sleeve and/or coating.

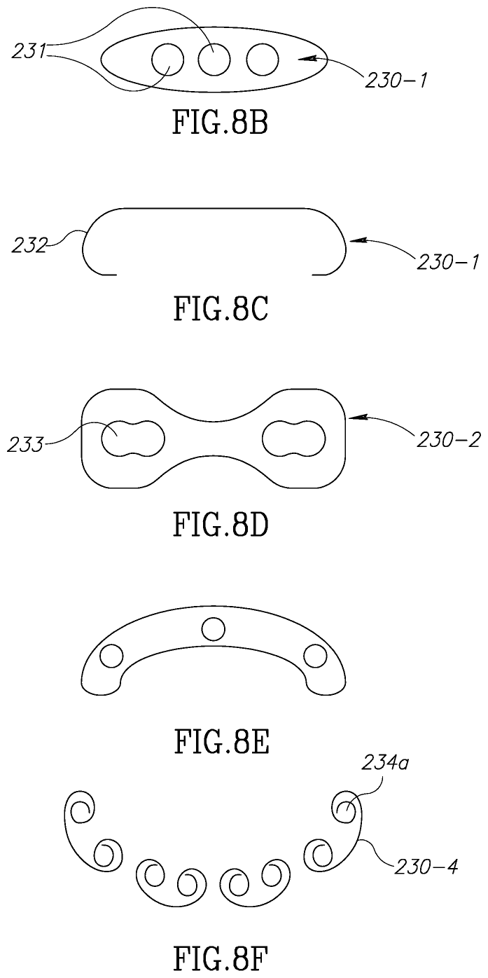

FIG. 8A schematically shows a CVS 107 comprising a plurality of leaflet staples 230 supporting optionally an anterior leaflet 44 of a mitral valve 43, in accordance with an embodiment of the disclosure. Staples 230 are optionally formed, as schematically shown in FIGS. 8B-8D, so that they may be connected in a daisy chain. Optionally, each leaflet staple 230 is formed having an eyelet 231 and a hook 232 at each end of the staple. Hook 232 of a given leaflet staple 230 may be configured to be bent to attach the leaflet staple to a cardiac valve leaflet such as leaflet 44 of mitral valve 43, and/or to be inserted through an eyelet 231 of an adjacent leaflet staple 230 to daisy chain the given leaflet staple with the adjacent staple. In FIG. 8A block arrows 318 point to hooks 232 that are inserted into eyelets 231 to daisy chain pairs of leaflet staples 230.

FIGS. 8B and 8C schematically show top and side views of an optionally oval shaped leaflet staple 230 designated leaflet staple 230-1 in accordance with embodiments of the disclosure. As shown in FIG. 8B leaflet staple 230-1 is formed having a plurality of, optionally three, eyelets 231 for receiving a hook of another leaflet staple 230 to daisy chain the staples. A side view of leaflet staple 230-1 schematically shown in FIG. 8C shows leaflet hooks 232 of a leaflet staple 230. Leaflet hooks 232 are configured to be inserted into eyelets of an adjacent leaflet staple 230 to daisy chain the leaflet staples and to puncture and curl to attach the leaflet staple to a region of leaflet 44. FIG. 8D schematically shows a top view of another leaflet staple 230-2 in accordance with an embodiment of the disclosure. Leaflet staple 230-2 has a dumbbell shaped silhouette and figure eight eyelets 233 for receiving staple hooks at different positions.

FIG. 8E schematically shows a semicircle staple, wherein a plurality of these staples may be configured to undulate, similar to the support braces in FIGS. 3A-3E. FIG. 8F are staples 230-4 with loops 234a formed at both ends of each staple and the loops further may be used to attach the staple to a leaflet. Loops 234a and the staples 230-4 are one continuous material. FIG. 8F schematically illustrates a plurality of wireform leaflet staples 230-4 formed having eyelets 234a. Eyelets 234a may be advantageous for attaching the staple to the leaflet. In an embodiment, eyelets 234a may be formed of shape memory material which may be used for shape deforming deployment of staples 230-4.

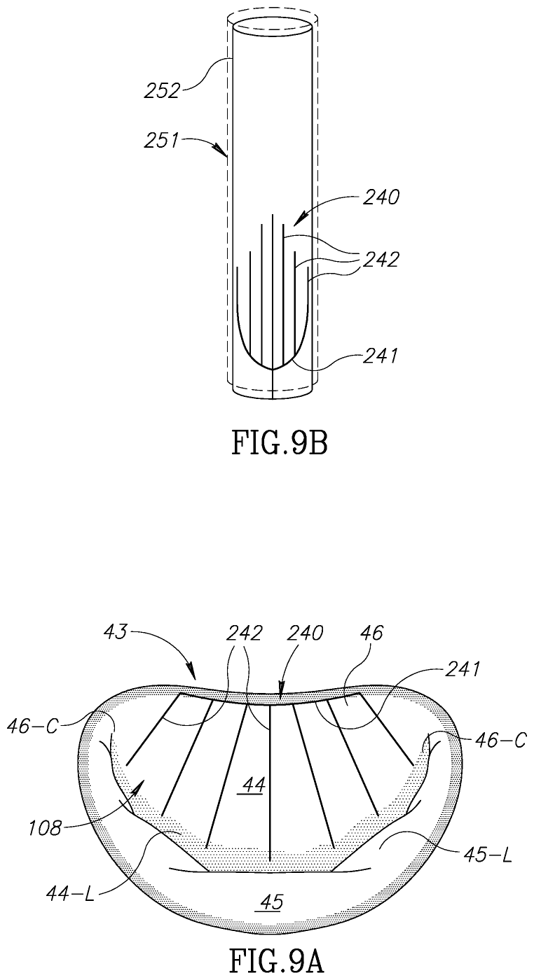

FIG. 9A schematically shows a CVS 108 comprising a comprising a spoke brace 240 having a backbone anchor 241 and a plurality of, optionally seven, spokes 242 optionally supporting an anterior leaflet 44 of a mitral valve 43, in accordance with an embodiment of the disclosure. FIG. 9B schematically shows spoke brace 240 folded to a delivery configuration in a delivery catheter 251 and mounted to an inner scaffolding tube 252 of the catheter for transcatheter deployment of the spoke brace on a cardiac valve.

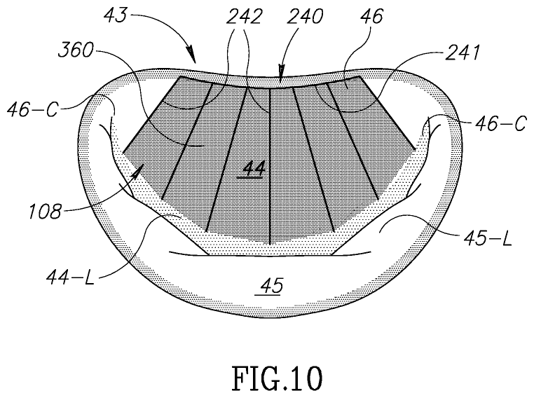

In an embodiment of the disclosure a CVS such as any of the CVSs shown in FIGS. 3A-9B may function as a scaffolding for a web, hereinafter also referred to as a leaflet web, made from a natural or artificial material that extends between at least portions of the leaflet support braces comprised in the CVS. The web provides additional support to a cardiac leaflet to which the CVS may be mounted and operates to conform the leaflet to a desired shape and associated functionality. FIG. 10, by way of example schematically shows CVS 240 in which spokes 242 function as a scaffolding for a leaflet web, schematically shown as hatching between the spokes that operates to improve functioning of anterior leaflet 44 of mitral valve 43.

There is therefore provided in accordance with an embodiment of the disclosure, apparatus for supporting a leaflet of a cardiac valve having an annulus and a plurality of leaflets extending from the annulus, the apparatus comprising: at least one support brace mountable to the cardiac valve and having: first and second ends each configured to be mounted to the cardiac valve in a region near a commissure of the cardiac valve associated with a leaflet of the plurality of leaflets; and a body configured to be attached to the leaflet and extend between the first and second ends along a lip of the leaflet. Optionally, the body is wire or ribbon shaped. Additionally, or alternatively the body comprises relatively thick segments connected by relatively short thin connecting segments.

In an embodiment the body comprises a plurality of staples configured to be connected in a daisy chain. Optionally, each staple comprises a hook configured to be bent to attach the staple to a cardiac valve leaflet. Optionally, each staple is formed having an eyelet operable to receive the hook of another staple to daisy chain the staples.

In an embodiment of the disclosure the body is formed having undulations. Optionally, the undulations exhibit substantially a constant spatial period along the length of the body. Optionally, the undulations exhibit a spatial period that varies along the length of the body. In an embodiment the undulations have substantially a same spatial amplitude. In an embodiment the undulations exhibit different spatial amplitudes. Optionally, the spatial amplitudes exhibit a maximum, and increasing amplitude with increase in distance from each end toward a location of the maximum.

In an embodiment the apparatus comprises a loop configured to be attached to the annulus of the valve. Optionally, an end of the first and second ends comprises an eyelet operable to be coupled to the loop to mount the end to the cardiac valve.

In an embodiment the apparatus an end of the first and second ends comprises an elastic member configured to be coupled to the annulus to attach the end to the annulus. Optionally, the elastic member comprises a coil spring.

In an embodiment the apparatus the apparatus comprises a plurality of ring loops configured to be attached to the leaflet and through which when attached to the leaflet a support brace of the at least one support brace is threadable to attach the support brace to the leaflet.

In an embodiment the apparatus the at least one support brace comprises at least one shape memory material tang configured to transform from a martensite state to an austenite state during which the tang curls to puncture and grip a region of tissue of the cardiac valve to mount the support brace to the cardiac valve. Optionally, the region of tissue comprises tissue in the annulus. Optionally, the region of tissue comprises tissue in the leaflet.

In an embodiment the apparatus the at least one support brace comprises a plurality of support braces. Optionally, the plurality of braces comprises first and second support braces configured to be attached respectively to first and second leaflets of the plurality of leaflets. Optionally, the first support brace is configured to extend beyond the second support brace on an antegrade side of the second support brace when both braces are attached to the first and second leaflets so that the second brace limits motion of the first brace when blood pressure operates to close the cardiac valve. Additionally, or alternatively the second support brace comprises a spur that extends beyond the first support brace on a retrograde side of the first support brace when both braces are mounted to the first and second leaflets and limits motion of the first support brace relative to the first support brace when blood pressure operates to close the cardiac valve.

There is further provided in accordance with an embodiment of the disclosure, apparatus for supporting a leaflet of a cardiac valve having an annulus and a plurality of leaflets extending from the annulus, the apparatus comprising: a backbone anchor having at least two ends configured to be mounted to the annulus of the cardiac valve; and a plurality of spokes that extend from the backbone anchor; wherein in a deployed configuration of the apparatus the spokes splay out to lie on and support a leaflet of the cardiac valve. Optionally, the apparatus comprises a web that extends between at least portions of the spokes. Additionally, or alternatively the apparatus has a delivery configuration in which delivery configuration the anchor and spokes are folded to fit between a delivery catheter and a scaffolding tube located inside the delivery catheter.

In the disclosure, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims as follows.

1. Apparatus for supporting a leaflet of a cardiac valve having an annulus and a plurality of leaflets extending from the annulus, the apparatus comprising:
at least one support brace mountable to the cardiac valve and having:
first and second ends each configured to be mounted to the cardiac valve in a region near a commissure of the cardiac valve associated with a leaflet of the plurality of leaflets; and
a body configured to be attached to the leaflet and extend between the first and second ends along a lip of the leaflet.

2. The apparatus according to claim 1 wherein the body is wire or ribbon shaped.

3. The apparatus according to claim 1 wherein the body comprises relatively thick segments connected by relatively short thin connecting segments.

4. The apparatus according to claim 1 wherein the body comprises a plurality of staples configured to be connected in a daisy chain.

5. The apparatus according to claim 4 wherein each staple comprises a hook configured to be bent to attach the staple to a cardiac valve leaflet.

6. The apparatus according to claim 5 wherein each staple is formed having an eyelet operable to receive the hook of another staple to daisy chain the staples.

7. The apparatus according to claim 2, wherein the body is formed having undulations.

8.-12. (canceled)

13. The apparatus according to claim 1 and comprising a loop configured to be attached to the annulus of the valve.

14. The apparatus according to claim 13, wherein an end of the first and second ends comprises an eyelet operable to be coupled to the loop to mount the end to the cardiac valve.

15. The apparatus according to claim 1, wherein an end of the first and second ends comprises an elastic member configured to be coupled to the annulus to attach the end to the annulus.

16. (canceled)

17. The apparatus according to claim 1 and comprising a plurality of ring loops configured to be attached to the leaflet and through which when attached to the leaflet a support brace of the at least one support brace is threadable to attach the support brace to the leaflet.

18.-21. (canceled)

22. The apparatus according to claim 1 comprising a plurality of support braces and further comprising at least one tying brace configured to span a distance between the plurality of support braces and attach to the support braces at a plurality of junctions.

23. The apparatus according to claim 22, wherein the plurality of braces comprises first and second support braces configured to be attached respectively to first and second leaflets of the plurality of leaflets.

24. The apparatus according to claim 23, wherein the first support brace is configured to extend beyond the second support brace on an antegrade side of the second support brace when both braces are attached to the first and second leaflets so that the second brace limits motion of the first brace when blood pressure operates to close the cardiac valve.

25. The apparatus according to claim 23, wherein the second support brace comprises a spur that extends beyond the first support brace on a retrograde side of the first support brace when both braces are mounted to the first and second leaflets and limits motion of the first support brace relative to the first support brace when blood pressure operates to close the cardiac valve.

26. Apparatus for supporting a leaflet of a cardiac valve having an annulus and a plurality of leaflets extending from the annulus, the apparatus comprising:
a backbone anchor having at least two ends configured to be mounted to the annulus of the cardiac valve; and
a plurality of spokes that extend from the backbone anchor;
wherein in a deployed configuration of the apparatus the spokes splay out to lie on and support a leaflet of the cardiac valve.

27. The apparatus according to claim 26 and comprising a web that extends between at least portions of the spokes.

28. The apparatus according to claim 26 wherein the apparatus has a delivery configuration in which delivery configuration the anchor and spokes are folded to fit between a delivery catheter and a scaffolding tube located inside the delivery catheter.
